(19) United States
(12) Patent Application Publication
(10) Pub. No.: US 2007/0207100 A1
(43) Pub. Date: Sep. 6, 2007

(54) Method for hair loss disguise

(76) Inventor: Carole J. Adams, Yakima, WA (US)

Correspondence Address: BLACK LOWE & GRAHAM, PLLC, 701 FIFTH AVENUE, SUITE 4800, SEATTLE, WA, 98104, US

(21) Appl. No.: 11/368,328

(22) Filed: Mar. 3, 2006

(51) Int. Cl. A61K 8/18

(52) U.S. Cl. 424/63

(57) ABSTRACT

The invention is directed to a process for disguising hair loss by selecting a cosmetic pigmentation to match the color of existing hair; disbursing the cosmetic pigmentation onto the scalp epidermis in the thinning hair area; and blending the cosmetic pigmentation to uniformly cover the scalp epidermis in the thinning hair area.

### PRIORITY CLAIM

This application claims priority to provisional application 60/657,955 filed Mar. 4, 2005, which is incorporated herein by reference.

### FIELD OF THE INVENTION

This invention relates generally to hair cosmetology and, more specifically, to cosmetics for disguising hair loss.

### BACKGROUND OF THE INVENTION

Thinning hair is a sign of medication usage, health- or genetically-related or of chemotherapy treatment for both men and women. It is an indicator of age-related balding. Thinning hair cannot adequately cover up the epidermis of the scalp. Thinning hair is caused by the shrinkage of the hair follicles that result in a reduction in hair thickness and visibility. In men, the thinning of hair results in a permanent receding hairline or baldness. In women, thinning hair occurs all over the head, but the frontal hairline is generally maintained.

People have used methods such as hair transplants, topically and internally applied hair growth medicine, hairpieces and hair weaves to treat or cover up thinning hair and baldness. These methods are often difficult to apply, time consuming to use, expensive or only partially effective. In addition, these methods are ill suited for dealing with areas of thinning hair.

Hair pieces and hair weaves may not be necessary for small areas of thinning hair. Moreover, hair pieces and hair weaves often look unnatural, and they may actually accelerate the rate of hair loss. Hair transplants are highly technical and costly procedures that can only be performed by trained professionals. Generally, hair transplants are more suited for individuals in advanced stages of hair loss. Topically and internally applied medicine, which must be taken regularly on an indefinite basis, are most effective in slowing the rate of hair loss. However, these medications usually do not restore the appearance of thinning areas because they seldom cause the re-growth of hair. Thus, an economical, easy to use, yet highly effective solution is needed for dealing with areas of thinning hair. This invention addresses these problems by providing a simple to apply, inexpensive and natural looking solution for concealing thinning hair areas.

### SUMMARY OF THE INVENTION

The present invention is directed to a method of disguising areas of thinning hair. Specifically, the present invention enhances the apparent fullness of thinning hair by coating the underlying scalp epidermis with cosmetic pigmentation. The method includes the steps of selecting a cosmetic pigmentation to match the color of existing hair, disbursing the selected cosmetic pigmentation into the thinning hair area, and blending the cosmetic pigmentation to uniformly cover the scalp epidermis in the area of thinning hair.

### BRIEF DESCRIPTION OF THE DRAWINGS

The patent application file contains at least one drawing executed in color. Copies of this patent application publication with color drawing(s) will be provided by the Office upon request and payment of the necessary fee.

Preferred and alternative embodiments of the present invention are described in detail below with reference to the following drawings.

FIG. 1 is a block diagram of a method according to the present invention;

FIGS. 2A-2D are photos of a user prior to using the method;

FIGS. 3A-3D are photos of the user of FIGS. 2A-2D after using the method.

### DETAILED DESCRIPTION

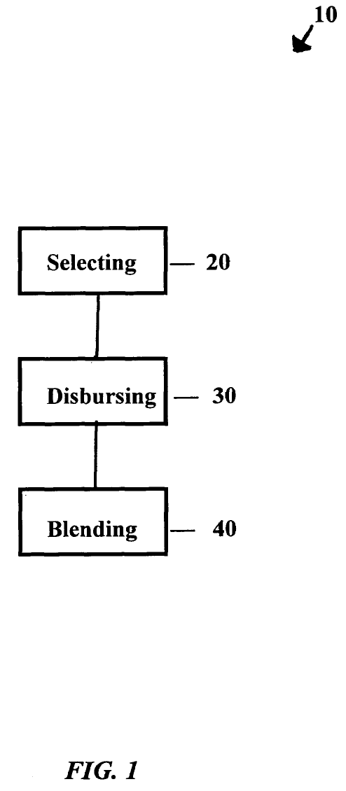

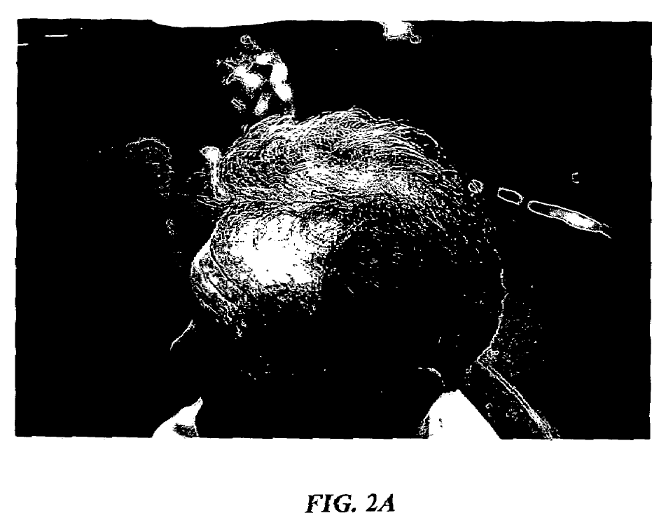

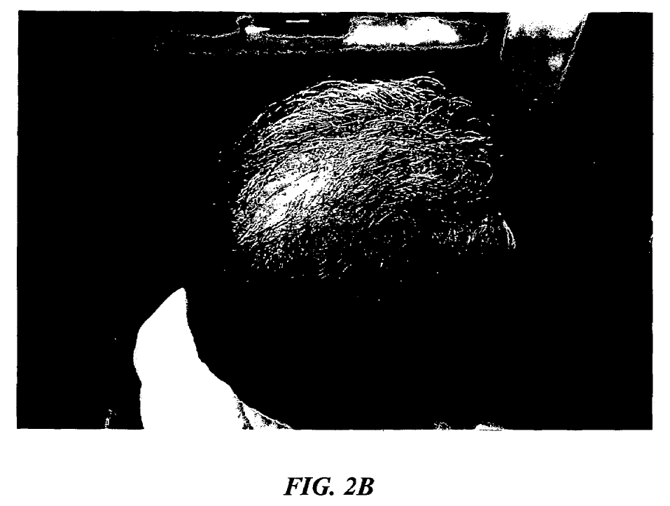

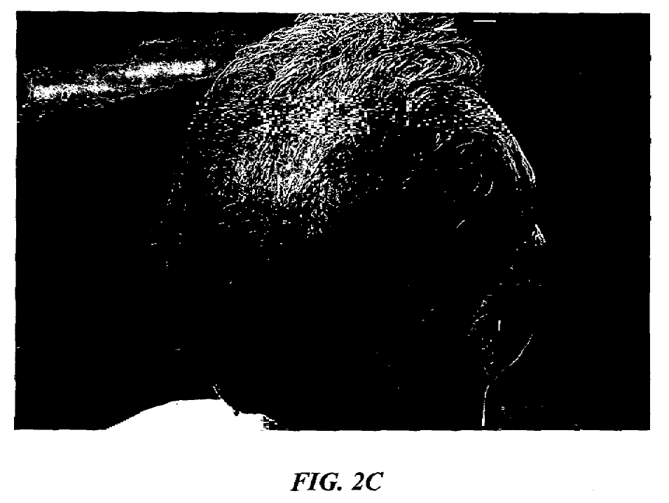

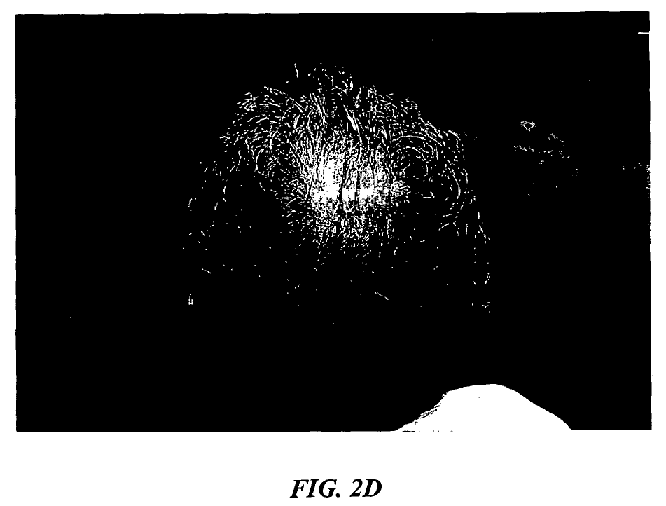

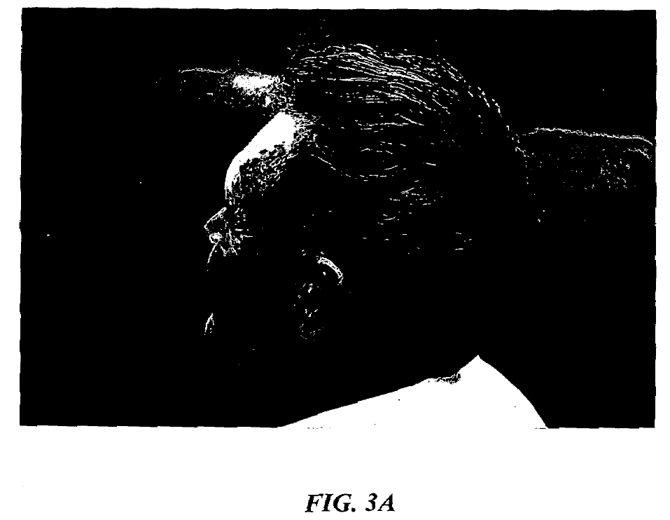

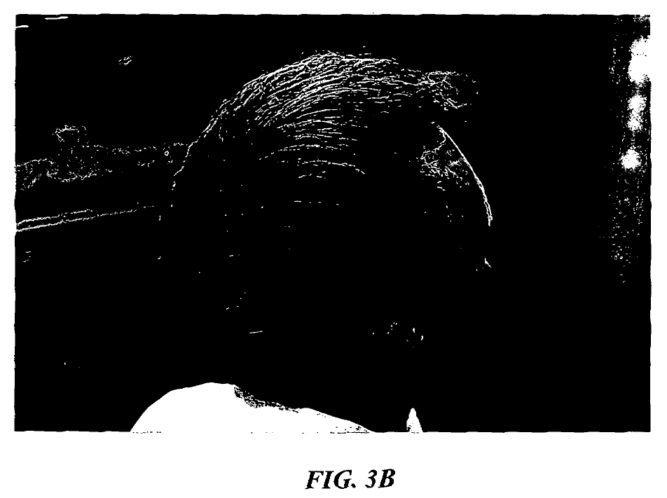

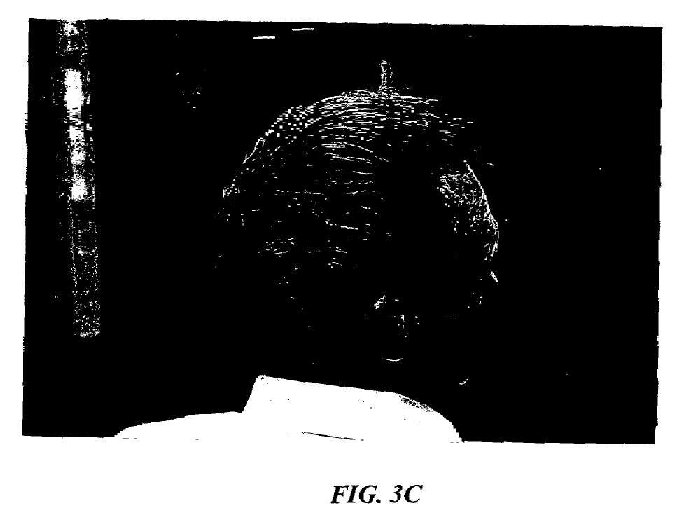

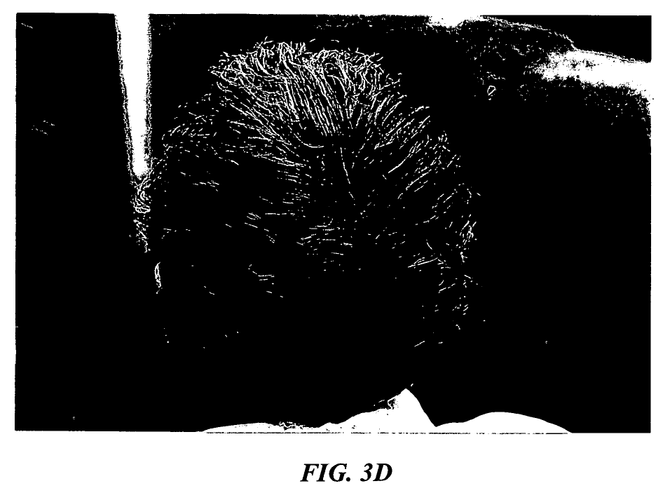

FIG. 1 shows a block diagram of a method according to the present invention for disguising hair loss. The method 10 includes the steps of selecting a cosmetic pigmentation to match a color of an area of thinning hair, shown at a block 20; disbursing the chosen cosmetic pigmentation to an exposed scalp epidermis of the thinning hair area, shown at a block 30; and, blending the cosmetic pigmentation on the epidermis to conceal the scalp and disguise hair loss, shown at a block 40.

In the selection step 20, cosmetics are selected. Presently preferred cosmetics include eye liner, eyebrow liner and eye shadow products, but the invention is not limited to these types of cosmetics. Any type of cosmetic, including body paint, dye, and hair dye, is included; indeed, anything used to cover and camouflage the scalp is included. The cosmetics employed include the following forms: cream, powder, soft and hard color pigmentation pencil, as well as other forms known to those having skill in the art. Alternatively, custom cosmetics specially formulated for application to the scalp may be used. The cosmetic is selected to closely approximate the overall color of the existing hair on the scalp.

In the disbursement step 30, the cosmetic pigmentation is applied to the scalp epidermis of the thinning hair area. Cosmetics in the form of soft or hard color pigmentation pencils are disbursed by drawing directly onto the scalp. Cosmetics in the form of cream or powder are applied with fingers. Cosmetics in the cream or pressed powder format are the easiest to disburse onto the scalp. The cosmetic pigmentation may be applied when the entire hair is dry, although the hair may be washed immediately prior to application.

In the blending step, the cosmetic pigmentation is blended with fingers, cosmetic sponge, cotton pad or other pliable material in order to smooth the cosmetic and color over the exposed scalp epidermis of the thinning hair areas.

The invention is used to provide a method of concealing areas of thinning hair. By approximating the color of existing hair on the scalp in the thinning hair areas, the invention serves to lessen the visual contrast between the exposed scalp, the thinning hair and the surrounding hair. The decreased visual contrast effectively disguises the hair loss.

The invention provides an easy to use, inexpensive yet effective solution to the problem of thinning hair. The process of selecting the proper color cosmetic, disbursing the cosmetic pigmentation on the scalp in the problematic thinning area, and blending the cosmetic pigmentation can be completed in approximately five minutes. The invention also provides flexibility. Since the cosmetic pigmentation is applied to the scalp, the hair in the thinning area may be styled, along with the rest of the hair, after application.

The process also provides a hair loss disguise that is both water resistant and transfer resistant. The application will stand up to moisture from perspiration, dampness from humidity and wetness from some exposure to rain. The application will not bleed or run in those situations. Nevertheless, the application can be removed with an ordinary cleanser, such as shampoo or soap, and water. The application of the cosmetic pigmentation to the scalp provides another benefit. A head covering, such as a hat or a scarf, may be worn after application without undesirable contact transfer of the cosmetic pigmentation, provided that the head covering is not pressed tightly against the application area.

Contact of hair with objects after application, such as with pillows, beddings and upholstery, also will not result in contact transfer of the cosmetic pigmentation. Because the process applies water resistant and transfer resistant cosmetic pigmentation to the scalp, the application is durable and will generally last up to several days under normal conditions. Overall, these characteristics demonstrate the efficacy of the invention.

In one embodiment, an eyebrow pencil of a particular color is selected to match the color of the existing hair. With the hair in a preferably dry condition, the eyebrow pencil is used to draw roughly parallel sagittal lines approximately quarter of an inch apart in the thinning hair area. The eyebrow pencil lines are then blended with fingers to evenly cover the thinning hair area to produce a natural looking result.

In another embodiment, after the application of the cosmetic pigmentation to the thinning hair area, the entire hair, including the thinning area, is styled with a curling iron, comb and hair spray.

While the preferred embodiment of the invention has been illustrated and described, as noted above, changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A method for disguising hair loss comprising:
selecting a cosmetic pigmentation to match a color of an area of thinning hair;
disbursing the selected cosmetic pigmentation into the thinning hair area; and
blending the cosmetic pigmentation to cover a portion of scalp in the thinning hair area.

2. The method of claim 1 wherein selecting includes selecting one or more cosmetic pigmentations.

3. The method of claim 1 wherein the cosmetic pigmentation includes at least one of eye liner, eyebrow liner, eye shadow, body paint, dye, and hair dye.

4. The method of claim 1 wherein the cosmetic pigmentation is in the form of at least one of cream, powder, soft color pigmentation pencil, hard color pigmentation pencil, or liquid.

5. The method of claim 1 wherein disbursing includes applying pigmentation to the scalp of the thinning hair area.

6. The method of claim 1 wherein disbursing includes drawing directly on the scalp with a pigmentation pencil.

7. The method of claim 1 wherein disbursing includes applying pigmentation with a finger.

8. The method of claim 1 wherein disbursing includes disbursing on the thinning hair area when the area is substantially dry.

9. The method of claim 1 wherein blending includes using at least one of a finger, a cosmetic sponge, and a cotton pad.

10. The method of claim 1 wherein blending includes smoothing the cosmetic pigmentation.

11. The method of claim 1 further including the step of removing the cosmetic pigmentation.

12. The method of claim 11 wherein removing includes using at least one of shampoo and soap.

13. The method of claim 6 wherein drawing includes drawing roughly parallel sagittal lines on the area.

14. The method of claim 9 wherein blending includes using a finger to blend eyebrow pencil lines.

15. The method of claim 1 wherein blending includes styling the hair with at least one of a curling iron, a comb, and a hairspray.

16. The method of claim 1 further including the step of evaluating the results of the selecting, disbursing, and blending steps, and repeating the steps if the evaluation is not satisfactory.
